SEQUENCE LISTING (18 sequences)

SEQ ID NO: 1 - DNA, 26 bp, Artificial Sequence
  oligonucleotide AS1411
ggtggtggtggttgtggtggtggtgg

SEQ ID NO: 2 - DNA, 14 bp, Artificial Sequence
  oligonucleotide GRO14A
gttgtttggggtgg

SEQ ID NO: 3 - DNA, 15 bp, Artificial Sequence
  oligonucleotide GRO15A
gttgtttggggtggt

SEQ ID NO: 4 - DNA, 25 bp, Artificial Sequence
  oligonucleotide GRO25A
ggttggggtgggtggggtgggtggg

SEQ ID NO: 5 - DNA, 28 bp, Artificial Sequence
  oligonucleotide GRO28A
tttggtggtggtggttgtggtggtggtg

SEQ ID NO: 6 - DNA, 29 bp, Artificial Sequence
  oligonucleotide GRO29A
tttggtggtggtggttgtggtggtggtgg

SEQ ID NO: 7 - DNA, 29 bp, Artificial Sequence
  oligonucleotide GRO29-2
tttggtggtggtggttttggtggtggtgg

SEQ ID NO: 8 - DNA, 29 bp, Artificial Sequence
  oligonucleotide GRO29-3
tttggtggtggtggtggtggtggtggtgg

SEQ ID NO: 9 - DNA, 29 bp, Artificial Sequence
  oligonucleotide GRO29-5
tttggtggtggtggtttgggtggtggtgg

SEQ ID NO: 10 - DNA, 13 bp, Artificial Sequence
  oligonucleotide GRO29-13
tggtggtggtggt

SEQ ID NO: 11 - DNA, 11 bp, Artificial Sequence
  oligonucleotide GRO11A
ggtggtggtgg

SEQ ID NO: 12 - DNA, 14 bp, Artificial Sequence
  oligonucleotide GRO14C
ggtggttgtggtgg

SEQ ID NO: 13 - DNA, 56 bp, Artificial Sequence
  oligonucleotide GRO56A
ggtggtggtggttgtggtggtggtggttgtggtggtggtggttgtggtggtggtgg

SEQ ID NO: 14 - DNA, 32 bp, Artificial Sequence
  oligonucleotide GRO32A
ggtggttgtggtggttgtggtggttgtggtgg

SEQ ID NO: 15 - DNA, 32 bp, Artificial Sequence
  oligonucleotide GRO32B
tttggtggtggtggttgtggtggtggtggttt

SEQ ID NO: 16 - DNA, 29 bp, Artificial Sequence
  oligonucleotide GRO29-6
ggtggtggtggttgtggtggtggtggttt

SEQ ID NO: 17 - DNA, 28 bp, Artificial Sequence
  oligonucleotide GRO28B
tttggtggtggtggtgtggtggtggtgg

SEQ ID NO: 18 - DNA, 10 bp, Artificial Sequence
  oligonucleotide GRO13A
tggtggtggt

(19) United States
(12) Patent Application Publication
(10) Pub. No.: US 2011/0105422 A1
(43) Pub. Date: May 5, 2011

(54) USE OF G-RICH OLIGONUCLEOTIDES FOR TREATING NEOPLASTIC DISEASES

(76) Inventors: Gary Acton, London (GB); Hakim Djeha, Hertfordshire (GB); Donna Dobinson, Hertfordshire (GB); Benjamin Doran, Hertfordshire (GB); David Halford Ashton Jones, Hertfordshire (GB)

(21) Appl. No.: 12/866,102

(22) PCT Filed: Feb. 5, 2009
(86) PCT No.: PCT/GB09/00326

§ 371 Date: Jan. 7, 2011

(30) Foreign Application Priority Data
Feb. 5, 2008 (GB) 0802075.2
May 16, 2008 (GB) 0808956.7

(51) Int. Cl. A61K 31/704; A61K 31/7088; A61P 35/00; A61P 35/02

(52) U.S. Cl. 514/34; 514/44 R

(57) ABSTRACT

The invention relates to methods of treating patients (either adult or paediatric) with tumours using G rich oligonucleotides. In embodiments of the invention, methods of treating patients with tumours using a combination of G rich oligonucleotides and a chemotherapeutic agent are provided. There are also provided pharmaceutical compositions and kits for use in the methods of the invention.

The present invention relates to materials and methods for the treatment of cancer. In particular, an aspect of the invention relates to a therapy comprising the administration of a G rich oligonucleotide in combination with a chemotherapeutic agent. An aspect of the invention relates to a therapy comprising the administration of G rich oligonucleotides for the treatment of paediatric cancer

Oligonucleotides have the potential to recognize unique sequences of DNA or RNA with a remarkable degree of specificity. For this reason they have been considered as promising candidates to realize gene specific therapies for the treatment of malignant, viral and inflammatory diseases. Two major strategies of oligonucleotide-mediated therapeutic intervention have been developed, namely, the antisense and antigene approaches.

The antisense strategy aims to down-regulate expression of a specific gene by hybridization of the oligonucleotide to the specific mRNA, resulting in inhibition of translation. Gewirtz et al. (1998) Blood 92, 712-736; Crooke (1998) Antisense Nucleic Acid Drug Dev. 8, 115-122; Branch (1998) Trends Biochem. Sci. 23, 45-50; Agrawal et al. (1998) Antisense Nucleic Acid Drug Dev. 8, 135-139.

The antigene strategy proposes to inhibit transcription of a target gene by means of triple helix formation between the oligonucleotide and specific sequences in the double-stranded genomic DNA. Helene et al. (1997) Ciba Found. Symp. 209, 94-102.

Whereas both the antisense and antigene strategies have met with some success, it has become clear in recent years that the interactions of oligonucleotides with the components of a living organism go far beyond sequence-specific hybridization with the target nucleic acid. Recent studies and re-examination of early antisense data have suggested that some of the observed biological effects of antisense oligonucleotides cannot be due entirely to Watson-Crick hybridization with the target mRNA. In some cases, the expected biological effect (e.g. inhibition of cell growth or apoptosis) was achieved, but this was not accompanied by a down regulation of the target protein and was thus unlikely to be a true antisense effect. White et al. (1996) Biochem. Biophys. Res. Commun. 227, 118-124; Dryden et al. (1998) J. Endocrinol. 157, 169-175.

In many cases, it was demonstrated that other non sequence specific oligonucleotides could exert biological effects that equalled or exceeded the antisense sequence. Barton et al. (1995) Br. J. Cancer 71,429437; Burgess et al. (1995) Proc. Natl. Acad. Sci. U.S.A. 92, 4051-4055; Benimetskaya et al. (1997) Nucleic Acids Res. 25, 2648-2656.

Though there is currently a high awareness among antisense investigators of the importance of appropriate control oligonucleotides, and the necessity of demonstrating inhibition of target protein production (Stein (1998) Antisense Nucleic Acid Drug Dev. 6, 129-132), the mechanism of non-antisense effects is poorly understood.

In particular, phosphodiester and phosphorothioate oligodeoxynucleotides containing contiguous guanosines (G) have been repeatedly found to have non-antisense effects on the growth of cells in culture.

Burgess et al. (1995) Proc. Natl. Acad. Sci. U.S.A. 92, 4051-4055; Benimetskaya et al. (1997) Nucleic Acids Res. 25, 2648-2656; Saijo et al. (1997) Jpn. J. Cancer Res. 88, 26-33. There is evidence that this activity is related to the ability of these oligonucleotides to form stable structures involving intramolecular or intermolecular G-quartets. Burgess et al. (1995) Proc. Natl. Acad. Sci. U.S.A. 92, 4051-4055; Benimetskaya et al. (1997) Nucleic Acids Res. 25, 2648-2656. G-quartets are square planar arrangements of four hydrogen-bonded guanines that are stabilized by monovalent cations.

Such structures are thought to play an important role in vivo and putative quartet forming sequences have been identified in telomeric DNA (Sundquist et al. (1989) Nature 342, 825-829), immunoglobulin switch region sequences (Sen et al. (1988) Nature 334, 364-366), HIV1 RNA (Sundquist et al. (1993) Proc. Natl. Acad. Sci. U.S.A. 90, 3393-3397), the fragile X repeat sequences (Fry et al (1994) Proc. Natl. Acad. Sci. U.S.A. 91, 4950-4954) and the retinoblastoma gene (Murchie et al. (1992) Nucleic Acids Res. 20, 49-53)

Applicants have previously described G-rich oligonucleotides (GROs) that have potent growth inhibitory effects that are unrelated to any expected antisense or antigene activity. The antiproliferative effects of these oligonucleotides have be identified by the applicants as being related to their ability to bind to a specific cellular protein. Because the GRO binding protein is also recognized by antinucleolin antibodies, Applicants have concluded that this protein is either nucleolin itself, or a protein of a similar size that shares immunogenic similarities with nucleolin.

Nucleolin is an abundant multifunctional 110 kDa phosphoprotein thought to be located predominantly in the nucleolus of proliferating cells (for reviews, see Tuteja et al. (1998) Crit. Rev. Biochem. Mol. Biol. 33, 407-436; Ginisty et al. (1999) J. Cell Sci. 112, 761-772). Nucleolin has been implicated in many aspects of ribosome biogenesis including the control of rDNA transcription, pre-ribosome packaging and organization of nucleolar chromatin. Tuteja et al. (1998) Crit. Rev. Biochem. Mol. Biol. 33, 407-436; Ginisty et al. (1999) J. Cell Sci. 112, 761-772; Ginisty et al. (1998) EMBO J. 17, 1476-1486.

Another role for nucleolin is as a shuttle protein that transports viral and cellular proteins between the cytoplasm and nucleus/nucleolus of the cell. Kibbey et al. (1995) J. Neurosci. Res. 42, 314-322; Lee et al. (1998) J. Biol. Chem. 273, 7650-7656; Waggoner et al. (1998) J. Virol. 72, 6699-6709.

Nucleolin is also implicated, directly or indirectly, in other roles including nuclear matrix structure (Gotzmann et al. (1997) Electrophoresis 18,26452653), cytokinesis and nuclear division (Léger-Silvestre et al. (1997) Chromosoma 105, 542-52), and as an RNA and DNA helicase (Tuteja et al. (1995) Gene 160, 143-148).

The multifunctional nature of nucleolin is reflected in its multidomain structure consisting of a histone-like N-terminus, a central domain containing RNA recognition motifs, and a glycine/arginine rich C-terminus. Lapeyre et al. (1987) Proc. Natl. Acad. Sci. U.S.A. 84, 1472-1476.

Levels of nucleolin are known to relate to the rate of cellular proliferation (Derenzini et al. (1995) Lab. Invest. 73, 497-502; Roussel et al. (1994) Exp. Cell Res. 214, 465-472.), being elevated in rapidly proliferating cells, such as malignant cells, and lower in more slowly dividing cells.

Chemotherapeutic agents are used in the treatment of Cancer. Topoisomerase II inhibitors comprise a group of useful chemotherapeutic agents which affect cell cycle progression during G²/M leading to G² arrest (Progress in Cell Cycle Research, Vol. 5, 295-300, 2003).

Doxorubicin (hydroxyldaunorubicin, also know as adriamycin) is a topoisomerase II inhibitor commonly used in the treatment of cancer, for example, leukaemia, Hodgkin's lymphoma, bladder cancer, breast cancer, stomach cancer, lung cancer, ovarian cancer, thyroid cancer, soft tissue sarcoma, and multiple myeloma. Doxorubicin is available commercially from e.g. Pharmacia under the name Adriamycin™ and is also available as a generic product. Doxorubicin is an anthracycline antibiotic with the chemical name of (8S,10S)-10-(4-amino-5-hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione:

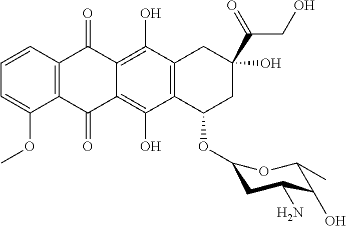

Doxorubicin is thought to interact with DNA by intercalation and to inhibit the activity of topoisomerase II. As a topoisomerase II inhibitor, it has been reported that doxorubicin induces DNA damage in G₂ phase cells (Carcinogenesis, Vol. 23, No. 3, 389-401, March 2002), and can trigger apoptosis of cells in the G₀-G₁ phases of the cell cycle (Cancer Research, 60, 1901-1907, Apr. 1, 2000).

Doxorubicin is typical of most chemotherapeutic agents in that it is not very selective in the targets it acts upon, thereby causing serious side-effects. Examples of side-effects of doxorubicin include nausea, vomiting, heart arrhythmias, neutropenia (a decrease in white blood cells), complete alopecia (hair loss) and serious cardiac side effects, including congestive heart failure, dilated cardiomyopathy, and death.

Paediatric cancers occur in approximately 1 in every 600 children under 15 years of age and are widely recognised as exhibiting different characteristics from cancers affecting adults. Paediatric cancers tend to occur in different parts of the body, have different histology and respond differently to treatment. Most paediatric cancers are treated using treatment regimes established for adult cancers. As such, there is a need to identify effective treatments for paediatric cancers that have been identified as effective against those paediatric conditions and not just used as an extrapolation of a related adult condition (Boklan (2006) Mol Cancer Ther 5(8): 1905-8; Balis (2000) Oncologis, 5:2-3).

The search for anti-cancer agents and methods of treatment with improved efficacy and reduced toxicity in different patient groups is ongoing and intense. The present invention seeks to provide further agents and methods for the treatment of cancers including paediatric cancers.

### SUMMARY OF THE INVENTION

G-rich oligonucleotides (GROs) as a monotherapy have demonstrated growth inhibition and/or consistent cell killing against various hematologic tumour cells. The inventors have identified that an anti-cancer effect can be obtained by means of treatment of sarcomas, blastomas, and lymphomas with a G rich oligonucleotide.

The inventors have also identified that an anti-Cancer effect can be obtained by means of a combined treatment with a G rich oligonucleotide, and a chemotherapeutic agent, such as a topoisomerase II inhibitor.

The inventors have also identified that a surprising anti-Cancer effect can be obtained by means of treatment of paediatric cancers with G rich oligonucleotides.

The term “topoisomerase II inhibitor”, as used herein, includes, but is not limited to, the anthracyclines, such as doxorubicin (hydroxyldaunorubicin, also known as adriamycin), doxorubicin liposomal formulation; daunorubicin, daunorubicin liposomal formulation; epirubicin, idarubicin and nemorubicin; the anthraquinones mitoxantrone and losoxantrone; and the podophillotoxines etoposide and teniposide.

The inventors have further identified that a synergistic anti-Cancer effect can be obtained by means of a combined treatment with a G rich oligonucleotide, and a chemotherapeutic agent, such as doxorubicin (hydroxyldaunorubicin, also known as adriamycin)

In a first aspect of the invention there is provided a pharmaceutical composition comprising a G rich oligonucleotide having the sequence of one of SEQ IDs Nos. 1 to 18 and a topoisomerase II inhibitor in conjunction with a pharmaceutically acceptable excipient, diluent or carrier.

Preferably, the topoisomerase II inhibitor is doxorubicin.

Examples of oligonucleotides of the present invention have the following nucleotide sequences:

AS1411-
(SEQ ID No: 1)
5′-GGTGGTGGTGGTTGTGGTGGTGGTGG-3′
(also known as GR026B and AGRO100)
GR014A-
(SEQ ID No: 2)
5′ GTTGTTTGGGGTGG-3′
GRO15A-
(SEQ ID No: 3)
5′-GTTGTTTGGGGTGGT-3′
GR025A-
(SEQ ID No: 4)
5′-GGTTGGGGTGGGTGGGGTGGGTGGG-3′
GR028A-
(SEQ ID No: 5)
5′-TTTGGTGGTGGTGGTTGTGGTGGTGGTG-3′
GR029A-
SEQ ID No: 6)
5′-TTTGGTGGTGGTGGTTGTGGTGGTGGTGG-3′
GR029-2-
(SEQ ID No: 7)
5′-TTTGGTGGTGGTGGTTTTGGTGGTGGTGG-3′
GR029-3-
(SEQ ID No: 8)
5′-TTTGGTGGTGGTGGTGGTGGTGGTGGTGG-3′
GR029-5-
(SEQ ID No: 9)
5′-TTTGGTGGTGGTGGTTTGGGTGGTGG TGG-3′
GR029-13-
(SEQ ID No: 10)
5′-TGGTGGTGGTGGT-3′
GRO11A-
(SEQ ID No: 11)
5′-GGTGGTGGTGG-3′
GRO14C-
(SEQ ID No: 12)
5′-GGTGGTTGTGGTGG-3′
GR056A-
(SEQ ID No: 13)
5′-GGTGGTGGTGGTTGTGGTGGTGGTGGTTGTGGTGGTGGTGGTTGTGG
TGGTGGTGG-3′
GR032A-
(SEQ ID No: 14)
5′-GGTGGTTGTGGTGGTTGTGGTGGTTGTGGTGG-3′
GR032B-
(SEQ ID No: 15)
5′-TTTGGTGGTGGTGGTTGTGGTGGTGGTGGTTT-3′
GR029-6-
(SEQ ID No: 16)
5′-GGTGGTGGTGGTTGTGGTGGTGGTGGTTT-3′
GR028B-
(SEQ ID No: 17)
5′-TTTGGTGGTGGTGGTGTGGTGGTGGTGG-3′
GR013A-
(SEQ ID No: 18)
5′-TGGTGGTGGT-3′.

Other oligonucleotides having the same activity are also contemplated.

By G-rich oligonucleotide (GRO) it is meant that the oligonucleotides consist of 4-100 nucleotides (preferably 10-30 nucleotides) with DNA, RNA, 2′-O-methyl, phosphorothioate or other chemically similar backbones. Their sequences contain one or more GGT motifs. The oligonucleotides have anti-proliferative activity against cells and bind to GRO binding protein and/or nucleolin. These properties can be demonstrated using techniques well known in the art such as an MTT assay or the EMSA technique (see WO 2000/61597).

The oligonucleotides of the present invention are rich in guanosine and are capable of forming G-quartet structures. Specifically, the oligonucleotides of the present invention are primarily comprised of thymidine and guanosine with at least one contiguous guanosine repeat in the sequence of each oligonucleotide.

As used herein, the term “oligonucleotide” is defined as a molecule comprising two or more deoxyribonucleotides or ribonucleotides. The exact size depends on a number of factors including the specificity and binding affinity to target ligands. In referring to “bases” or “nucleotides” the terms include both deoxyribonucleic acids and ribonucleic acids.

Preferably, the G-rich oligonucleotide has the sequence of SEQ ID 1.

In one embodiment, the G-rich oligonucleotide has a 3′ end and a 5′ end, and one or both of the 3′ and 5′ ends have been modified to alter a property of the G-rich oligonucleotide.

The oligonucleotides can be modified at their 3′ end in order to alter a specific property of the oligonucleotide. For example, the 3′-terminus of the oligonucleotide can be modified by the addition of a propylamine group which has been found to increase the stability of the oligonucleotide to serum nucleases. Other modifications that are well known in the art include 3′ and 5′ modifications, for example, the binding of cholesterol, and backbone modifications, for example, phosphorothioate substitution and/or 2′-O-methyl RNA.

In a second aspect of the invention there is provided a kit of parts comprising:
a G-rich oligonucleotide having the sequence of one of SEQ IDs Nos. 1 to 18 in a therapeutically effective amount;
(ii) doxorubicin in a therapeutically effective amount; and
(iii) instructions for their use.

By “therapeutically effective amount” we mean an amount of an oligonucleotide of the present invention or chemotherapeutic agent such as doxorubicin, that when administered to the subject either alone or in combination with another agent, ameliorates a symptom of the disease, disorder, or condition, such as by inhibiting or reducing the proliferation of dysplastic, hyperproliferative, or malignant cells. The therapeutically effective amount may be empirically determined by a skilled person such as a clinician based on the patient's clinical parameters including, but not limited to the stage of disease, age, gender, histology, and likelihood for tumour recurrence.

Optionally the kit further comprises:
(iv) means for administering the G-rich oligonucleotide and/or doxorubicin to a patient

Conveniently, the G-rich oligonucleotide and doxorubicin are provided separately.

Alternatively, the G-rich oligonucleotide and doxorubicin are provided as an admixture.

Preferably, the G-rich oligonucleotide has the sequence of SEQ ID 1.

In one embodiment, the G-rich oligonucleotide has a 3′ end and a 5′ end, and one or both of the 3′ and 5′ ends have been modified to alter a property of the G-rich oligonucleotide.

Preferably the G-rich oligonucleotide has the sequence of SEQ ID 1.

In one embodiment, the G-rich oligonucleotide has a 3′ end and a 5′ end, and one or both of the 3′ and 5′ ends have been modified to alter a property of the G-rich oligonucleotide.

In a third aspect of the invention there is provided a method for inhibiting the proliferation of malignant, dysplastic, and/or hyperproliferative cells, said method comprising administering to the subject a therapeutically effective amount of a G-rich oligonucleotide having the sequence of one of SEQ IDs Nos. 1 to 18 in combination with the chemotherapeutic agent doxorubicin.

In certain embodiments of the invention, the combination of a G-rich oligonucleotide having the sequence of one of SEQ IDs Nos. 1 to 18 with the chemotherapeutic agent doxorubicin is synergistic.

As used herein, the terms “synergy,” “synergism,” and “synergistic” relate to the coordinated action of two or more chemotherapeutic agents with a more than expected additive effect.

By “in combination with one another” regarding the G-rich oligonucleotide and chemotherapeutic agent treatments we include the meaning not only that the G-rich oligonucleotide and chemotherapeutic agents are administered simultaneously, but also that they are administered separately and sequentially.

Preferably the G-rich oligonucleotide and chemotherapeutic agents are administered between 0 and 24 hours apart with either the oligonucleotide or the chemotherapeutic being administered first.

The inhibition may be an in vitro or an in vivo method.

The term “inhibiting the proliferation of malignant, dysplastic, and/or hyperplastic cells” includes any partial or total growth inhibition and includes decreases in the rate of proliferation or growth of the cells.

As used herein, the term “neoplastic” includes the new, abnormal growth of tissues and/or cells, such as a cancer or tumour, including, for example, breast cancer, leukaemia or prostate cancer. The term “neoplastic” also includes malignant cells which can invade and destroy adjacent structures and/or metastasize.

As used herein, the term “dysplastic” includes any abnormal growth of cells, tissues, or structures including conditions such as psoriasis.

The term “subject” means all animals including humans. Examples of subjects include humans, cows, dogs, cats, goats, sheep, and pigs. The term “patient” means a subject having a disorder in need of treatment.

Subjects can be adult or paediatric subjects. A human paediatric individual is a human individual at any age between the day of its birth (i.e, zero (0) years of age) and 21 years of age. A human paediatric individual includes a “neonate” or “newborn” which is a human individual at any age between the day of its birth (i.e., zero (0) years of age) and 30 days of age; an “infant” which is a human individual at any age between 31 days and two years of age; a “child” which is an individual at any age between two and twelve years of age; an “adolescent” which is an individual at any age between twelve and twenty-one years of age. A human adult is an individual older than twenty-one years of age.

Those skilled in the art are easily able to identify patients having a malignant, dysplastic, or a hyperproliferative condition such as a cancer or psoriasis, respectively.

In one embodiment, the administration of the G-rich oligonucleotide precedes treatment with the chemotherapeutic agent.

In a second embodiment, the chemotherapeutic agent treatment precedes treatment with the G-rich oligonucleotide.

In a third embodiment, both the G-rich oligonucleotide and the chemotherapeutic agent are administered simultaneously.

Preferably, the G-rich oligonucleotide has the sequence of SEQ ID 1.

In one embodiment, the G-rich oligonucleotide has a 3′ end and a 5′ end, and one or both of the 3′ and 5′ ends have been modified to alter a property of the G-rich oligonucleotide.

Advantageously the malignant, dysplastic, and/or hyperproliferative cells are associated with a disorder selected from: acute myelogenous leukaemia, acute myeloid leukaemia (AML), acute lymphoblastic leukaemia (ALL), chronic myelogenous leukemia (CML), lymphomas, non-Hodgkin's lymphoma, Wilm's tumour, neuroblastoma, soft tissue and bone sarcomas, breast carcinoma, ovarian carcinoma, bladder carcinoma, pancreas carcinoma, thyroid carcinoma, gastric cancer, renal cancer, Hodgkin's disease, malignant lymphoma, bronchiogenic carcinoma, paediatric cancers, basal cell carcinoma, melanoma, acute promyelocytic leukaemia, myelodysplastic syndrome, chronic lymphocytic leukemia, rhabdomyosarcoma; osteosarcoma; medulloblastoma; craniopharyngioma; retinoblastoma; Ewing's sarcoma; lymphomas; non-Hodgkin's lymphoma; and Hodgkin's lymphoma and solid tumours including squamous cell carcinoma (such as head and neck cancer, and/or squamous cell carcinoma of the head and neck).

In a fourth aspect of the invention there is provided a method for treating a disease characterised by malignant, dysplastic, and/or hyperproliferative cells comprising exposing the malignant, dysplastic, and/or hyperproliferative cells to a combination of a G-rich oligonucleotide having the sequence of one of SEQ IDs Nos. 1 to 18 and the chemotherapeutic agent doxorubicin; wherein the G-rich oligonucleotide and the chemotherapeutic agent are administered in combination with one another.

By “treatment” we include the meanings that the number of malignant, dysplastic, and/or hyperproliferative cells is reduced and/or further malignant, dysplastic, and/or hyperproliferative cell growth is retarded and/or prevented and/or the malignant, dysplastic, and/or hyperproliferative cells are killed. Malignant, dysplastic, and/or hyperproliferative cells are characteristic of tumours and of Cancers.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

Preferably the G-rich oligonucleotide has the sequence of SEQ ID 1.

In one embodiment, the G-rich oligonucleotide has a 3′ end and a 5′ end, and one or both of the 3′ and 5′ ends have been modified to alter a property of the G-rich oligonucleotide.

Advantageously the malignant, dysplastic, and/or hyperproliferative cells are associated with a disorder selected from: acute myelogenous leukaemia, acute myeloid leukaemia (AML), acute lymphoblastic leukaemia (ALL), chronic myelogenous leukemia (CML), lymphomas, non-Hodgkin's lymphoma, Wilm's tumour, neuroblastoma, soft tissue and bone sarcomas, breast carcinoma, ovarian carcinoma, bladder carcinoma, pancreas carcinoma, thyroid carcinoma, gastric cancer, renal cancer, Hodgkin's disease, malignant lymphoma, bronchiogenic carcinoma, paediatric cancers, basal cell carcinoma, melanoma, acute promyelocytic leukaemia, myelodysplastic syndrome, chronic lymphocytic leukemia, rhabdomyosarcoma; osteosarcoma; medulloblastoma; craniopharyngioma; retinoblastoma; Ewing's sarcoma; lymphomas; non-Hodgkin's lymphoma; and Hodgkin's lymphoma and solid tumours including squamous cell carcinoma (such as head and neck cancer, and/or squamous cell carcinoma of the head and neck).

The GROs of the present invention can be administered to a patient or subject either alone or as part of a pharmaceutical composition. The GROs can be administered to patients either orally, rectally, parenterally (intravenously, intramuscularly, or subcutaneously), intracistemally, intravaginally, intraperitonally, intravesically, locally (powders, ointments, or drops), or as a buccal or nasal spray.

In a fifth aspect of the invention there is provided a combination of a G-rich oligonucleotide having the sequence of one of SEQ IDs Nos. 1 to 18 and the chemotherapeutic agent doxorubicin for use as a medicament.

In a sixth aspect of the invention there is provided a use of a combination of a G-rich oligonucleotide having the sequence of one of SEQ IDs Nos. 1 to 18 and the chemotherapeutic agent doxorubicin in the manufacture of a medicament for treating a disease characterised by malignant, dysplastic, and/or hyperproliferative cells.

In a seventh aspect of the invention there is provided a combination of a G-rich oligonucleotide having the sequence of one of SEQ IDs Nos. 1 to 18 and the chemotherapeutic agent doxorubicin for use in the treatment of a disease characterised by malignant, dysplastic, and/or hyperproliferative cells.

In any of the fifth, sixth and seventh aspects of the invention, preferably the G-rich oligonucleotide has the sequence of SEQ ID 1.

Further preferably, the G-rich oligonucleotide has a 3′ end and a 5′ end, and one or both of the 3′ and 5′ ends have been modified to alter a property of the G-rich oligonucleotide.

Advantageously, in any of the fifth, sixth and seventh aspects of the invention the malignant, dysplastic, and/or hyperproliferative cells are associated with at least one of the following disorders: acute myelogenous leukaemia, acute myeloid leukaemia (AML), acute lymphoblastic leukaemia (ALL), chronic myelogenous leukemia (CML), lymphomas, non-Hodgkin's lymphoma, Wilm's tumour, neuroblastoma, soft tissue and bone sarcomas, breast carcinoma, ovarian carcinoma, bladder carcinoma, pancreas carcinoma, thyroid carcinoma, gastric cancer, renal cancer, Hodgkin's disease, malignant lymphoma, bronchiogenic carcinoma, paediatric cancers, basal cell carcinoma, melanoma, acute promyelocytic leukaemia, myelodysplastic syndrome, chronic lymphocytic leukemia, rhabdomyosarcoma; osteosarcoma; medulloblastoma; craniopharyngioma; retinoblastoma; Ewing's sarcoma; lymphomas; non-Hodgkin's lymphoma; and Hodgkin's lymphoma and solid tumours including squamous cell carcinoma (such as head and neck cancer. and/or squamous cell carcinoma of the head and neck).

In certain embodiments of the invention, the malignant, dysplastic, and/or hyperproliferative cells are associated with Burkitt's lymphoma.

In some embodiments of the invention, the malignant, dysplastic, and/or hyperproliferative cells are associated with acute myelogenous leukaemia or acute myeloid leukaemia (AML),

In certain embodiments of the invention, a G-rich oligonucleotide having the sequence of one of SEQ IDs Nos. 1 to 18 potentiates the activity of the chemotherapeutic agent doxorubicin.

In an additional aspect of the invention there is provided a G-rich oligonucleotide having the sequence of one of SEQ IDs Nos. 1 to 18 for use in the treatment of a disease characterised by malignant, dysplastic, and/or hyperproliferative cells associated with a sarcoma, a blastoma, or a lymphoma.

In another aspect of the invention there is provided a use of a G-rich oligonucleotide having the sequence of one of SEQ IDs Nos. 1 to 18 in the manufacture of a medicament for treating a cancer selected from a sarcoma, a blastoma, or a lymphoma.

In certain embodiments wherein a G-rich oligonucleotide having the sequence of one of SEQ IDs Nos. 1 to 18 is useful for treating or manufacturing a medicament for the treatment of cancer, the sarcoma, blastoma, or lymphoma is selected from neuroblastoma; rhabdomyosarcoma; osteosarcoma; medulloblastoma; craniopharyngioma; retinoblastoma; Ewing's sarcoma; and Burkitt's lymphoma.

In certain embodiments of the invention, a G-rich oligonucleotide having the sequence of one of SEQ IDs Nos. 1 to 18 is administered to a patient or subject to treat malignant, dysplastic, and/or hyperproliferative cells associated with Burkitt's lymphoma, neuroblastoma; rhabdomyosarcoma; or osteosarcoma.

In a further aspect of the invention there is provided a kit of parts comprising:
(i) a G-rich oligonucleotide having the sequence of one of SEQ IDs Nos. 1 to 18 in a therapeutically effective amount;
(ii) instructions for their use in a paediatric cancer patient with a cancer selected from neuroblastoma; rhabdomyosarcoma; osteosarcoma; medulloblastoma; craniopharyngioma; retinoblastoma; Ewing's sarcoma; and Burkitt's lymphoma.

Preferably the kit also comprises
(iii) means for administering the G-rich oligonucleotide to a paediatric cancer patient.

Preferably the G-rich oligonucleotide has the sequence of SEQ ID 1.

In one embodiment, the G-rich oligonucleotide has a 3′ end and a 5′ end, and one or both of the 3′ and 5′ ends have been modified to alter a property of the G-rich oligonucleotide.

Examples embodying an aspect of the invention will now be described with reference to the following figures in which:

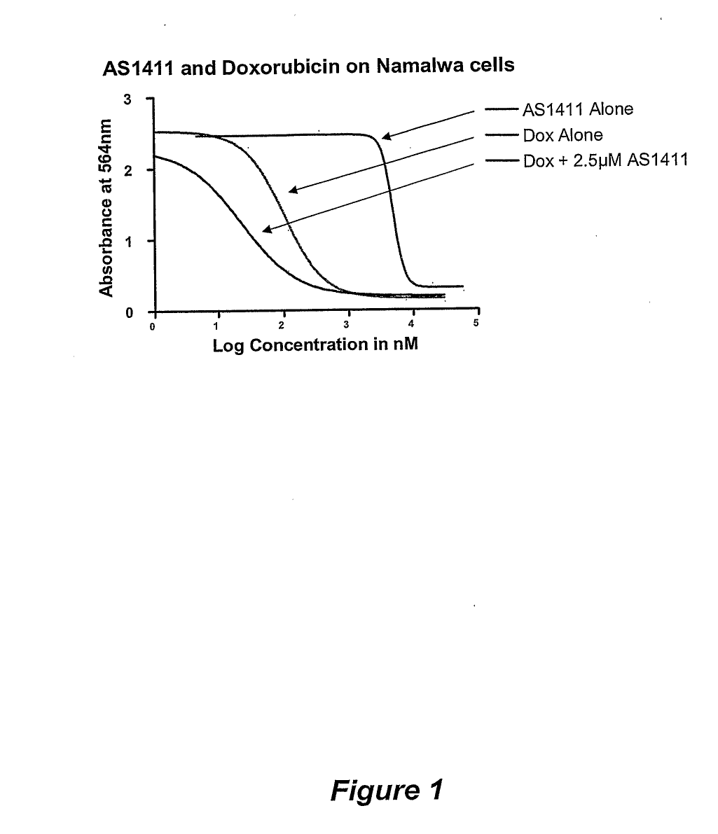

FIG. 1—AS1411 and Doxorubicin on Namalwa cells

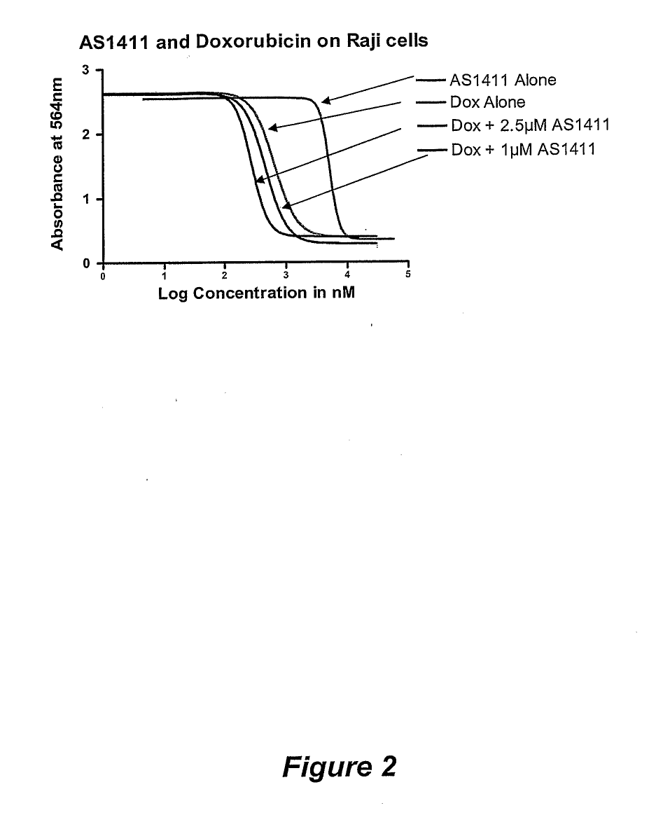

FIG. 2—AS1411 and Doxorubicin on Raji cells

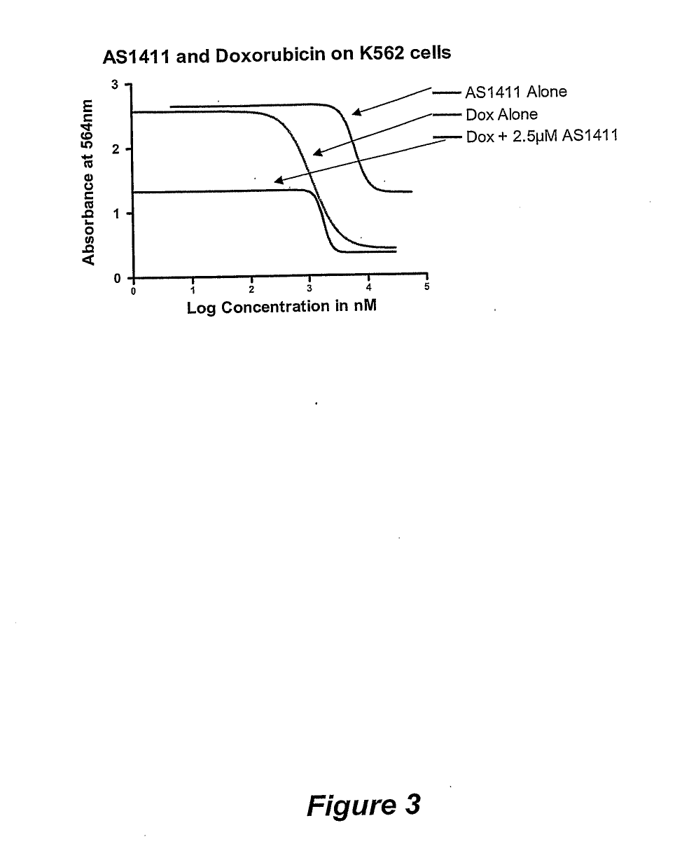

FIG. 3—AS1411 and Doxorubicin on K562 cells

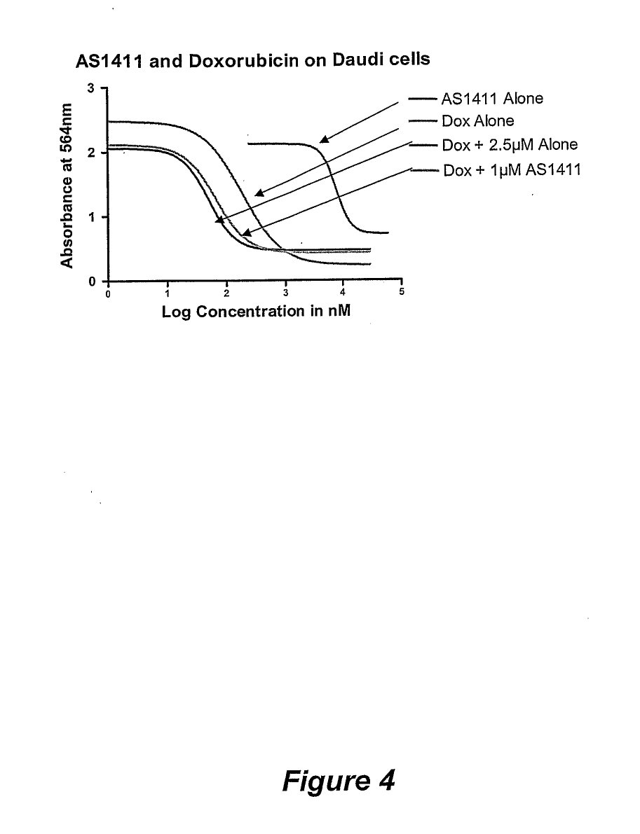

FIG. 4—AS1411 and Doxorubicin on Daudi cells

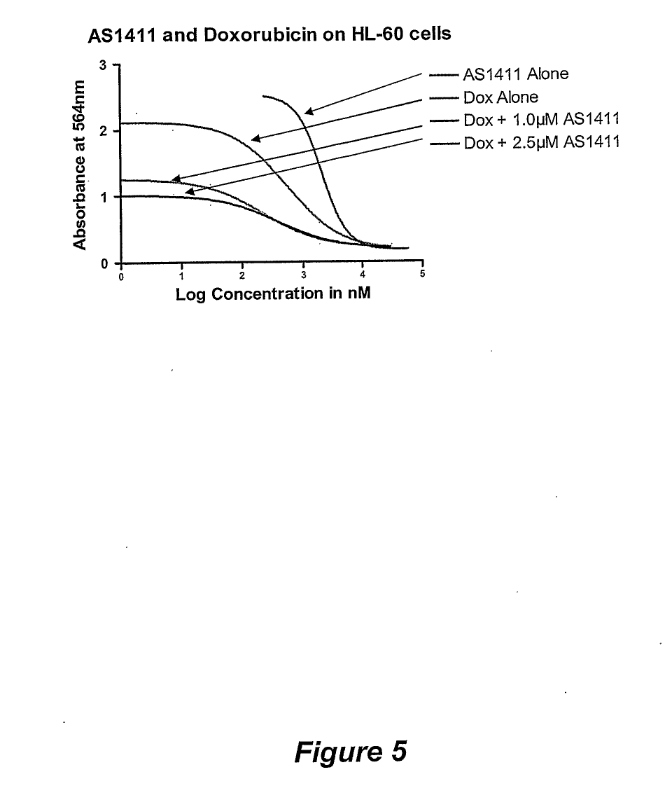

FIG. 5—AS1411 and Doxorubicin on HL-60 cells

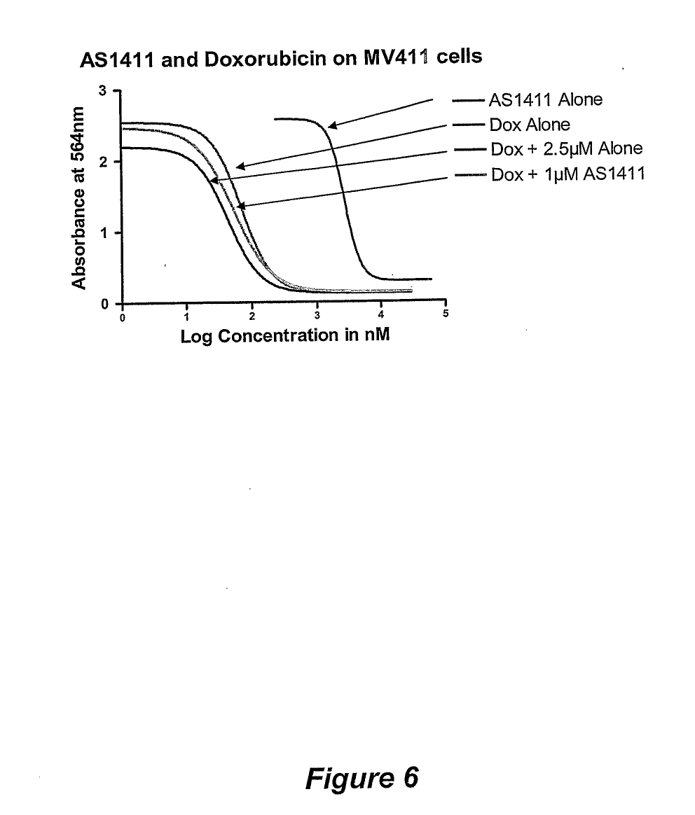

FIG. 6—AS1411 and Doxorubicin on MV411 cells

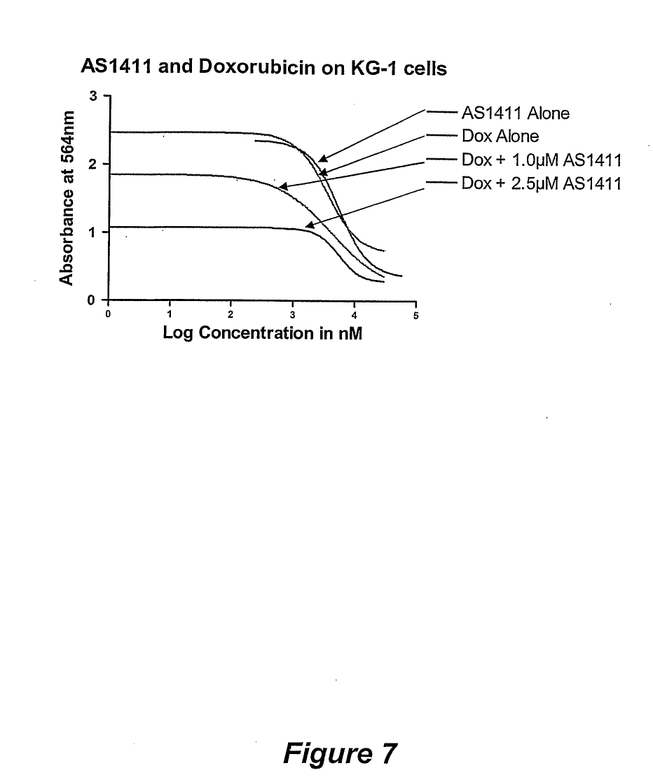

FIG. 7—AS1411 and Doxorubicin on KG-1 cells

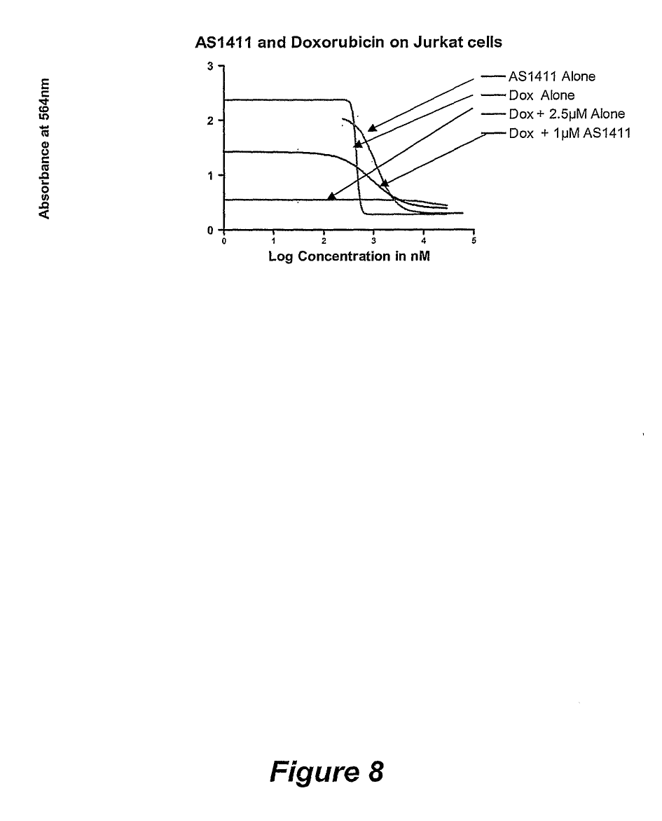

FIG. 8—AS1411 and Doxorubicin on Jurkat cells

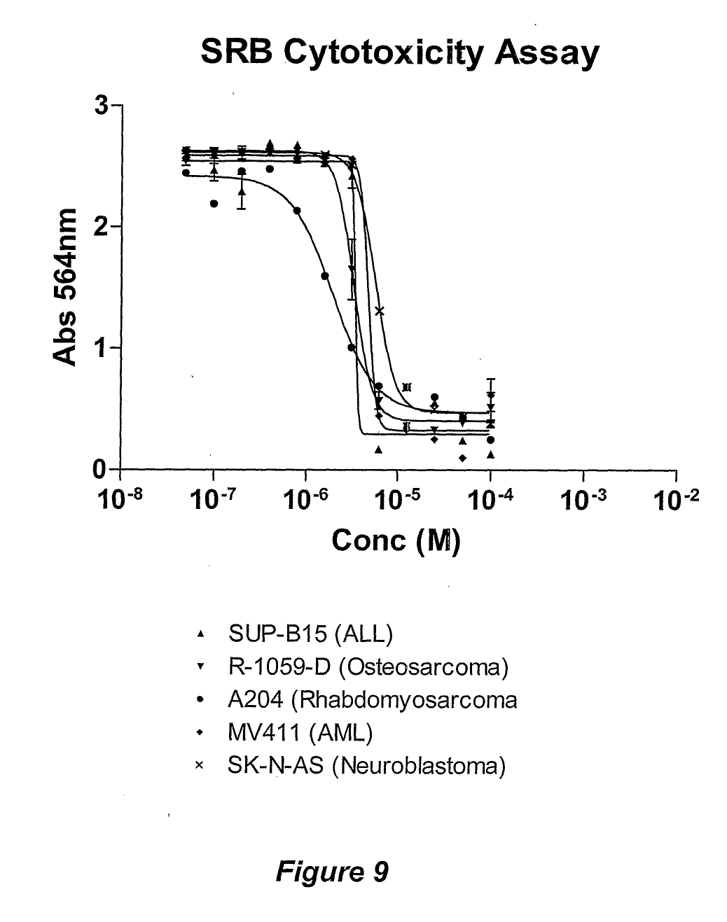

FIG. 9: SRB assay

SRB assay showing example data from paediatric cell line assays. Paediatric cell lines exhibit similar IC₅₀ values when exposed to AS1411 for a 6-day assay

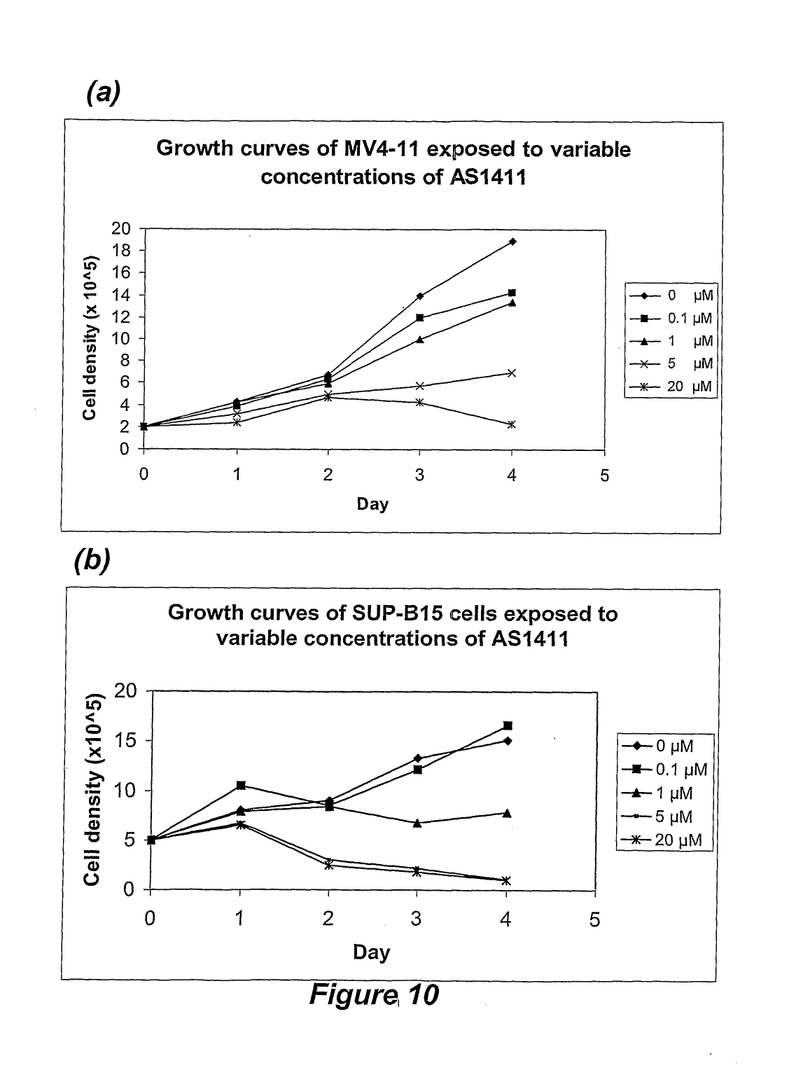

FIG. 10. Cytostatic effects

Cell growth of MV4-11 (AML) and SUP-B15 (ALL) cells exposed to AS1411

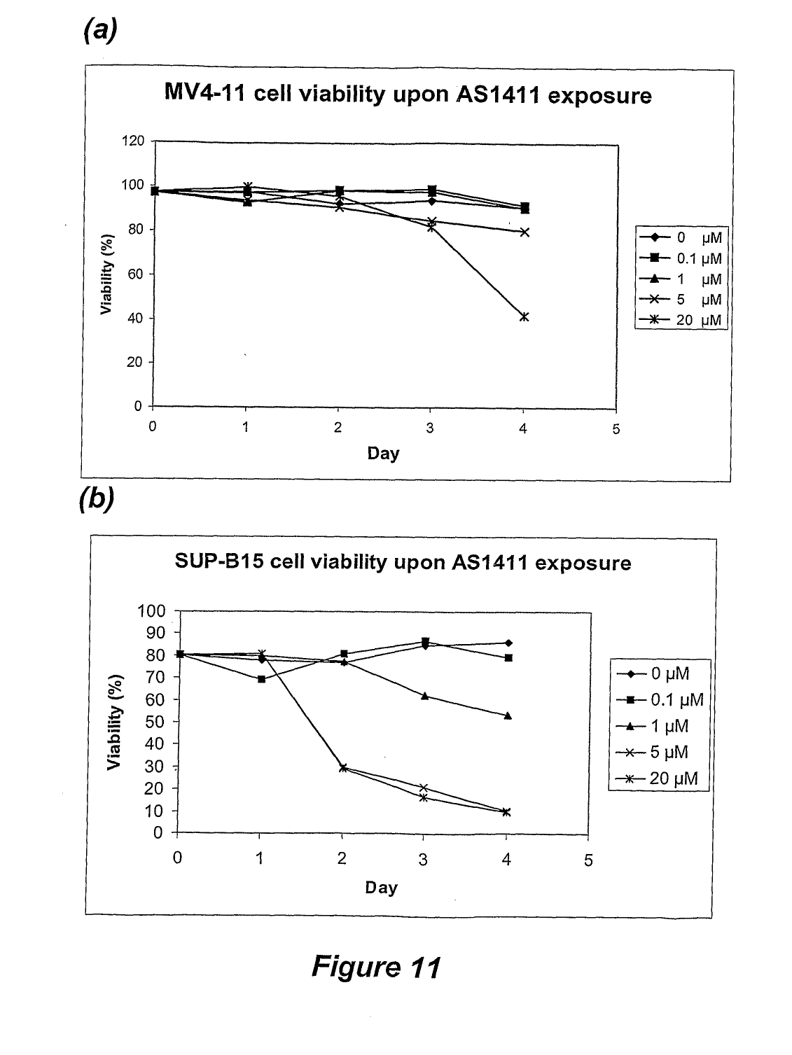

FIG. 11: Cytotoxic effects

Viability of MV4-11 (AML) and SUP-B15 (ALL) cells exposed to AS1411

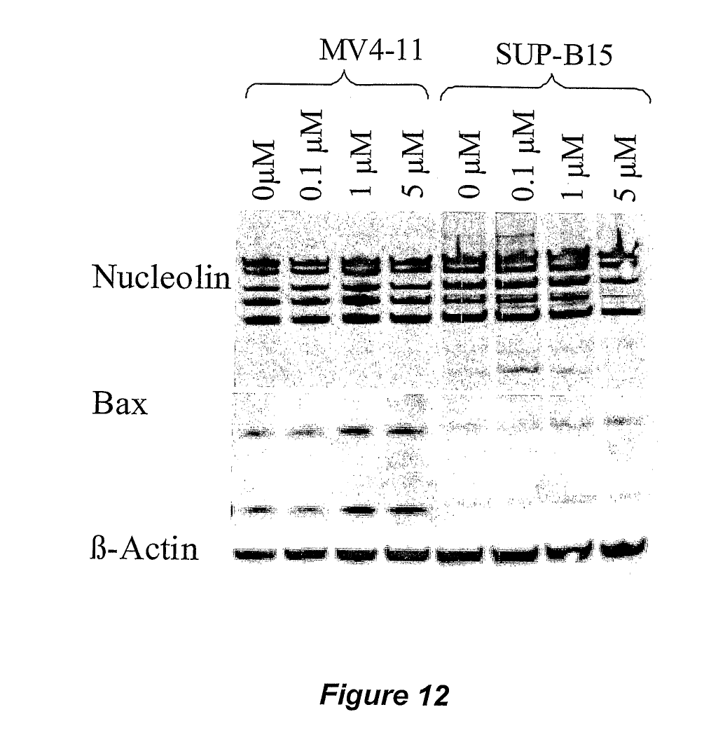

FIG. 12: Western blot analysis

Western blot analysis showing increased levels of Bax protein upon AS1411 exposure

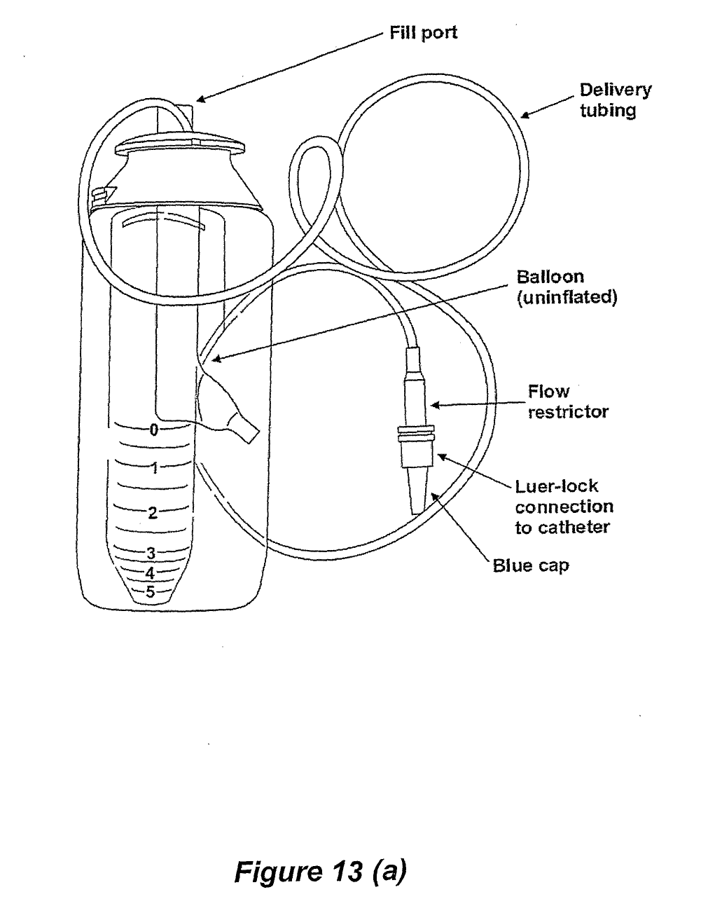

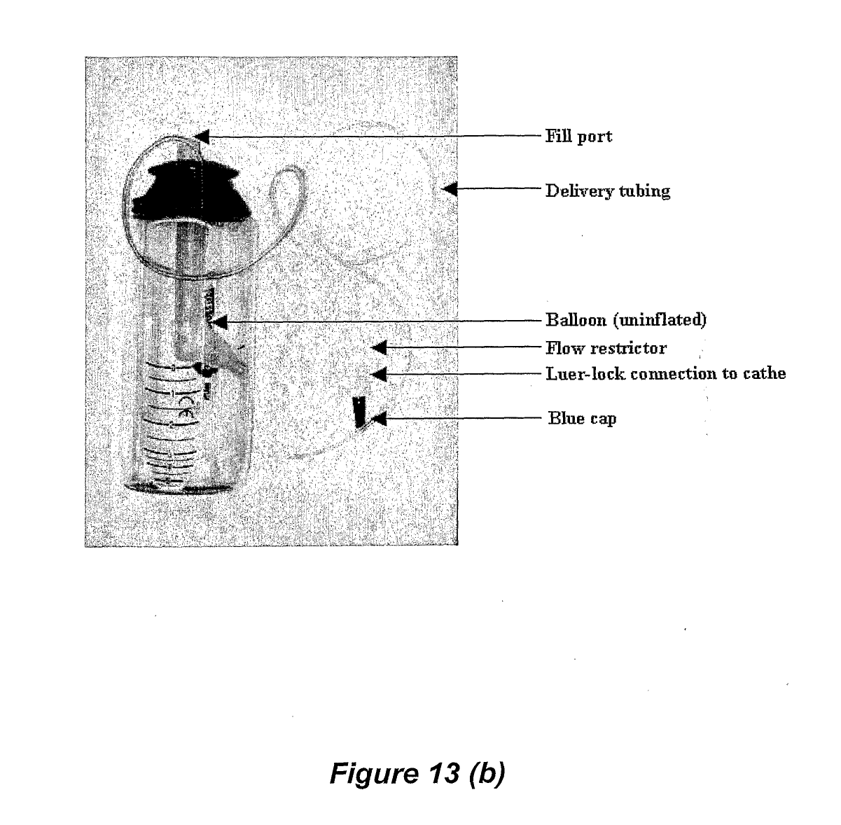

FIG. 13—Baxter FOLFusor LV10 device (a) shows line representation and (b) shows photograph of device.

### EXAMPLE 1

### Effects of AS1411 (GRO SEQ ID No. 1) and Doxorubicin

### Cell Lines Used

The cell lines used (and their properties) are described in Table 1 below.

TABLE 1
Cell lines

| | Origin | | | | Other |
|---|---|---|---|---|---|
| Cell Line | Organ | Cell Type | Disease | Immunology | Information |
| HL-60 | Peripheral | Promyeloblast | Acute | CD3− | Pseudodiploid. |
| | Blood | | Promyelocytic | CD4+ | Expresses |
| | | | Leukaemia | CD13+ | complement. |
| | | | (AML; FAB M2) | CD14− | Expresses Fc. |
| | | | | CD15+ | Amplified c- |
| | | | | CD19− | myc gene. |
| | | | | CD33+ | |
| | | | | CD34− | |
| | | | | HLA-DR− | |
| K562 | Bone | Chronic | Chronic | CD3− | Triploid. |
| | Marrow | Myeloid | Myelogenous | CD7+ | Haemoglobin |
| | (Pleural | Leukemia | Leukemia | CD13+ | Synthesis. |
| | Effusion) | (in blast crisis) | (CML) | CD19− | Philadelphia |
| | | | | CD34− | chromosome+. |
| | | | | CD41(+) | |
| | | | | CD42+ | |
| | | | | CD45+ | |
| | | | | CD71+ | |
| KG-1 | Bone | Myeloblast | Erythro- | CD3− | Near-diploid. |
| | Marrow | | leukemia | CD4− | EBNA −ve. |
| | (Pleural | | leading to | CD13+ | |
| | Effusion) | | Acute | CD14− | |
| | | | Myelogenous | CD15+ | |
| | | | Leukemia | CD19− | |
| | | | (AML) | CD33+ | |
| | | | | CD34+ | |
| | | | | HLA-DR+ | |
| MV-4-11 | — | Myeloblast | Biphenotypic B | CD3− | Hyperdiploid |
| | | | Acute Myelo- | CD4(+) | karyotype. |
| | | | monocytic | CD5− | FLT3 internal |
| | | | Leukemia (AML | CD8− | tandem |
| | | | FAB M5) | CD10− | duplication. |
| | | | | CD13+ | |
| | | | | CD14− | |
| | | | | CD15+ | |
| | | | | CD19− | |
| | | | | CD21− | |
| | | | | CD25− | |
| | | | | CD33+ | |
| | | | | CD34+ | |
| | | | | CD37− | |
| | | | | CD68+ | |
| | | | | CD138− | |
| | | | | HLA-DR+ | |
| Daudi | Peripheral | B Lymphoblast | Burkitt | CD3− | Near diploid |
| | Blood | | Lymphoma | CD10+ | karyotype with |
| | | | | CD13− | 20% polyploidy. |
| | | | | CD19+ | Produces IgM |
| | | | | CD20+ | (kappa light |
| | | | | CD34− | chain). |
| | | | | CD37+ | Expresses |
| | | | | CD38+ | complement. |
| | | | | CD79a+ | Expresses Fc. |
| | | | | cyCD79a+ | Surface Ig |
| | | | | CD80+ | (slg) +ve |
| | | | | CD138− | β-2− |
| | | | | HLA-DR+ | microglobulin −ve. |
| | | | | sm/cyIgG− | EBNA +ve. |
| | | | | sm/cyIgM+ | EBV +ive. |
| | | | | sm/cykappa+ | Express mRNA |
| | | | | sm/cylambda− | for bcl-2. |
| Namalwa | Tumour | B Lymphoblast | Burkitt | CD3− | Hypodiploid |
| | Mass | | Lymphoma | CD10+ | karyotype with |
| | | | | CD13− | 4% polyploidy. |
| | | | | CD19+ | Produces IgM |
| | | | | CD20+ | (lambda light |
| | | | | CD34− | chain). |
| | | | | CD37− | EBV +ve. |
| | | | | CD79a+ | |
| | | | | CD80− | |
| | | | | CD138+ | |
| | | | | HLA-DR+ | |
| | | | | sm/cyIgM+ | |
| | | | | sm/cyIgG− | |
| | | | | sm/cykappa− | |
| | | | | sm/cylambda+ | |
| Raji | Left Maxilla | B Lymphoblast | Burkitt | CD3− | Flat-moded |
| | | | Lymphoma | CD10+ | hypotetraploid |
| | | | | CD13− | karyotype with |
| | | | | CD19+ | 12% polyploidy. |
| | | | | CD20+ | |
| | | | | CD34− | |
| | | | | CD37+ | |
| | | | | CD79a+ | |
| | | | | CD80+ | |
| | | | | CD138− | |
| | | | | HLA-DR+ | |
| | | | | smIgG(+) | |
| | | | | cyIgG− | |
| | | | | smIgM(+) | |
| | | | | cyIgM+ | |
| | | | | sm/cykappa− | |
| | | | | sm/cylambda− | |

### Sulphorhodamine B Assay

Cells of the types described above were seeded in wells of a 96-well plate at a number optimised for each cell line.

TABLE 2
Cells Seeded

| Cell Line | Density |
|---|---|
| Namalwa | 5000 |
| MV411 | 3000 |
| KG-1 | 7500 |
| K562 | 3000 |
| Daudi | 10000 |
| Raji | 5000 |
| HL-60 | 10000 |

A fixed concentration of AS1411 (either 1 μM or 2.5 μM) was added with varying concentrations of doxorubicin (ranging between 0.12 nM and 30667.0 nM) and cells were incubated for 6 days. A control was run with varying amounts of AS1411 without Doxorubicin. A second control series was run with the varying amounts of Doxorubicin but with no AS1411 present.

Cells were then washed, fixed to the 96-well plate with 16% TCA and exposed to the dye Sulphorhodamine B (SRB; available from Sigma-Aldrich, Dorset, UK; catalogue number S-1402). The optical density of the remaining cell mass after exposure to AS1411 was measured in a microplate spectrophotometer and IC₅₀ determined.

The experiments were run in duplicate and the mean optical density calculated.

Combination index (CI) is determined using Calcusyn software (Biosoft, Cambridge UK) which employs the method of Chou, T.-C. and Talalay, P (See, Chou et al, Adv. Enz. Regul. 22: 27-55, 1984; and Chou, T.-C., Pharmacological Reviews 58:621-681, 2006.).

### Results

The results (as shown in Tables 3-10 below and FIGS. 1-9) demonstrate the synergistic effect of administering doxorubicin in conjunction with a G rich oligonucleotide.

TABLE 3
Namalwa (FIG. 1)

| | | | | IC50 | | CI Value | Concentration |
|---|---|---|---|---|---|---|---|
| Drug | n | Mean IC50 | | Range | | Range | Range (nM) |
| Doxorubicin | 2 | 69.68 | nM | 37.02-102.33 | nM | — | — |
| AS1411 | 2 | 3.2 | μM | 2.11-4.21 | μM | — | — |
| Doxorubicin + | 2 | 17.17 | nM | 11.79-22.54 | nM | 0.222-0.281 | 29.9-479.1 |
| 2.5 μM | | | | | | | |
| AS1411 | | | | | | | |

TABLE 4
MV411 (FIG. 6)

| | | | | IC50 | | CI Value | Concentration |
|---|---|---|---|---|---|---|---|
| Drug | n | Mean IC50 | | Range | | Range | Range (nM) |
| Doxorubicin | 2 | 75.87 | nM | 69.77-81.97 | nM | — | — |
| AS1411 | 2 | 2.36 | μM | 1.90-2.81 | μM | — | — |
| Doxorubicin + | 2 | 35.12 | nM | 26.81-43.42 | nM | 0.235-0.715 | 29.9-1916.7 |
| 1.0 μM | | | | | | | |
| AS1411 | | | | | | | |

| | | | | | | |
|---|---|---|---|---|---|---|
| Doxorubicin + | 1 | 53.08 | nM | — | 0.235-0.750 | 29.9-1916.7 |
| 2.5 μM | | | | | | |
| AS1411 | | | | | | |

TABLE 5
KG-1 (FIG. 7)

| | | | | IC50 | | CI Value | Concentration |
|---|---|---|---|---|---|---|---|
| Drug | n | Mean IC50 | | Range | | Range | Range (nM) |
| Doxorubicin | 2 | 4860.89 | nM | 3452.36-6269.41 | nM | — | — |
| AS1411 | 2 | 2.32 | μM | 1.72-2.92 | μM | — | — |
| Doxorubicin + | 2 | 4692.08 | nM | 4068.12-5316.04 | nM | 0.105-0.411 | 1916.7-30667.0 |
| 1.0 μM | | | | | | | |
| AS1411 | | | | | | | |
| Doxorubicin + | 2 | 6977.20 | nM | 5322.96-8631.44 | nM | 0.136-0.846 | 0.47-30667.0 |
| 2.5 μM | | | | | | | |
| AS1411 | | | | | | | |

TABLE 6
K562 (FIG. 3)

| | | | | IC50 | | CI Value | Concentration |
|---|---|---|---|---|---|---|---|
| Drug | n | Mean IC50 | | Range | | Range | Range (nM) |
| Doxorubicin | 2 | 1255.4 | nM | 1123.25-1387.55 | nM | — | — |
| AS1411 | 2 | 7.03 | μM | 6.87-7.19 | μM | — | — |
| Doxorubicin + | 2 | 1722.0 | nM | 1672.94-1771.06 | nM | 0.186-0.472 | 0.47-7667.0 |
| 2.5 μM | | | | | | | |
| AS1411 | | | | | | | |

TABLE 7
Daudi (FIG. 4)

| | | | | IC50 | | CI Value | Concentration |
|---|---|---|---|---|---|---|---|
| Drug | n | Mean IC50 | | Range | | Range | Range (nM) |
| Doxorubicin | 2 | 219.54 | nM | 182.06-257.01 | nM | — | — |
| AS1411 | 2 | 9.35 | μM | 7.75-10.95 | μM | — | — |
| Doxorubicin + | 2 | 108.09 | nM | 66.83-149.34 | nM | 0.108-0.736 | 1.87-1916.7 |
| 1.0 μM | | | | | | | |
| AS1411 | | | | | | | |
| Doxorubicin + | 2 | 57.16 | nM | 48.12-66.19 | nM | 0.105-0.797 | 29.9-1916.7 |
| 2.5 μM | | | | | | | |
| AS1411 | | | | | | | |

TABLE 8
Raji (FIG. 2)

| | | | | IC50 | | CI Value | Concentration |
|---|---|---|---|---|---|---|---|
| Drug | n | Mean IC50 | | Range | | Range | Range (nM) |
| Doxorubicin | 2 | 746.07 | nM | 657.11-835.02 | nM | — | — |
| AS1411 | 2 | 4.94 | μM | 4.54-5.34 | μM | — | — |
| Doxorubicin + | 2 | 444.31 | nM | 420.74-467.87 | nM | 0.24-0.85 | 479.1-7667.0 |
| 1.0 μM | | | | | | | |
| AS1411 | | | | | | | |
| Doxorubicin + | 2 | 326.58 | nM | 277.99-375.17 | nM | 0.031-0.809 | 0.12-30667.0 |
| 2.5 μM | | | | | | | |
| AS1411 | | | | | | | |

TABLE 9
HL-60 (FIG. 5)

| | | | | IC50 | | CI Value | Concentration |
|---|---|---|---|---|---|---|---|
| Drug | n | Mean IC50 | | Range | | Range | Range (nM) |
| Doxorubicin | 2 | 638.52 | nM | 547.65-729.39 | nM | — | — |
| AS1411 | 2 | 2.62 | μM | 2.13-3.11 | μM | — | — |
| Doxorubicin + | 2 | 540.99 | nM | 215.72-866.25 | nM | 0.230-0.710 | 119.80-7667.0 |
| 1.0 μM | | | | | | | |
| AS1411 | | | | | | | |
| Doxorubicin + | 2 | 638.85 | nM | 379.84-897.85 | nM | 0.255-0.701 | 29.9-7667.0 |
| 2.5 μM | | | | | | | |
| AS1411 | | | | | | | |

TABLE 10
Jurkat (FIG. 8)

| | | | | | | Concen- |
|---|---|---|---|---|---|---|
| | | | | IC50 | CI Value | tration |
| Drug | n | Mean IC50 | | Range | Range | Range (nM) |
| Doxorubicin | 1 | 426.07 | nM | — | — | — |
| AS1411 | 1 | 1.131 | μM | — | — | — |
| Doxorubicin + | 1 | 909.93 | nM | — | 0.265-0.725 | 0.47-1916.7 |
| 1.0 μM | | | | | | |
| AS1411 | | | | | | |

| | | | | | |
|---|---|---|---|---|---|
| Doxorubicin + | 1 | — | — | — | — |
| 2.5 μM | | | | | |
| AS1411 | | | | | |

Inhibition of growth in vitro with AS1411 in combination with doxorubicin of the human Burkitt's lymphoma cell lines NAMALWA (Table 3, FIG. 1, 5000 cells/well), Daudi (Table 7, FIG. 4, 10000 cells/well) and Raji (Table 8, FIG. 2, 5000 cells/well) observed using fixed, non-toxic concentration of AS1411 (2.5 μM) applied with increasing concentrations of doxorubicin indicate a synergistic effect against various Burkitt's lymphoma lines when AS1411 and doxorubicin were combined (n=2) based on the numerical data presented in Tables 3, 7, and 9. Combination Index analysis at concentrations on the steepest part of the curve resulted in values of 0.6, 0.3 and 0.6 for NAMALWA, Daudi and Raji cells, respectively.

TABLE 11
Synergistic effect

| | Doxorubicin IC₅₀ (nm) | | |
|---|---|---|---|
| | | In combination with 2.5 | Fold increase in |
| Cell Line | Alone | μM AS1411 | potency |
| NAMALWA | 69.7 | 17.2 | 4.1 |
| Daudi | 219.5 | 57.2 | 3.8 |
| Raji | 746.1 | 326.6 | 2.3 |

Table 11 above demonstrates the synergistic effect that doxorubicin and AS1411 have when administered in conjunction with one another.

### EXAMPLE 2

### Use of Combination Therapy in Cancer Treatment

The combination therapy experimentally tested in example 1 can be applied to use in the treatment of human tumours.

Treatment of human tumours requires administration of the standard clinical chemotherapy dose in mg/m² (mg/m² is calculated approximately by multiplying mg/kg by 37) for the chemotherapeutic agent being used. The standard clinical dose for a particular patient can easily be calculated based on that patient's specific circumstances and would form part of the day to day activities of the skilled person.

The time between administration of the chemotherapeutic agent and the G rich oligonucleotide is preferably between 0 and 24 hours, with either the chemotherapeutic or the G rich oligonucleotide being administered first. It is well within the skilled person's capabilities to construct a schedule of times for administering the chemotherapeutic and G rich oligonucleotide based on the needs of the patient and availability of appropriate resources.

The combination therapy will be administered in a course of treatment. The exact frequency of treatment administration within the course and length of the course as a whole will depend upon the particular chemotherapeutic agent being used and the circumstances of the individual patient. It is entirely within the scope of a skilled person's abilities to be able to determine the appropriate length and frequency of treatment.

### EXAMPLE 3

### Administration of Combination Therapy in Cancer Treatment Using an Intravenous Infusion

AS1411 is given to patients via intravenous infusion over a period of 7 days. The daily amount to be administered to the patient is calculated based on dose in mg/kg and the patient weight.

Fresh solutions are prepared on each infusion day, by diluting AS1411 drug product into 5% dextrose within an infusion bag (alternatives to dextrose include any known infusion system such as saline). Appropriate infusion bags are known to those skilled in the art. A fresh infusion bag is preferably prepared at the start of each 24-hour period. After calculation of the required dose of AS1411, an equivalent volume of dextrose should be removed from the bag, and the required dose of AS1411 added directly to the bag for a total final volume of 500 mL.

Once prepared, infusion bags containing AS1411 can be stored at +2° C. to +5° C. until administration. Drug can be prepared up to 6 hours prior to dosing.

Reconstituted AS1411 in 5% dextrose is administered at room temperature as soon as possible following reconstitution. The appropriate dose of AS1411 is administered as a 500 ml intravenous infusion. Infusion of AS1411 is as close to 24 hours as possible, accounting for changing of infusion bags, or clotting of infusion lines.

Doxorubicin is given to patients at a dose of 60-75 mg/m² as a single agent and 40-60 mg/m² as a intravenous infusion every 21 to 28 days. Therefore suitable doses are between 40 and 75 mg/m². Preparation of doxorubicin is performed following supplier's instructions.

### EXAMPLE 4

### G Rich Oligonucleotide Effect on Paediatric Cancer Cell Lines

### Methods

Cell Culture: Cells were cultured in T75 flasks and cell counts performed using the trypan blue dye exclusion method (whereby sterile Trypan blue solution 0.4% (e.g. Sigma T-8154) is added to cell cultures and non-viable cells are unable to exclude the dye and hence appear blue).

### Sulphorhodamine B Assay

Cells were typically seeded in wells of a 96-well plate as follows for each cell line:

| | |
|---|---|
| R-1059-D | 500 |
| A204 | 1000 |
| SK-N-AS | 4000 |
| MC/CAR | 10,000 |
| SUP-B15 | 10,000 |
| MV4-11 | 5000 |

AS1411 (G rich oligonucleotide of sequence ID No. 1) was added at a concentration selected from 0, 0.1, 1, 5 or 20 M and cells were incubated for 6 days

Cells were then washed, fixed to the 96-well plate and exposed to the dye Sulphorhodamine B (SRB; available from Sigma-Aldrich, Dorset, UK; catalogue number S-1402. The optical density of the remaining cell mass after exposure to AS1411 was measured in a microplate spectrophotometer and IC₅₀ determined. For the time course experiments, AS1411 was washed off cells at the stated time-point, and then fresh medium applied to the cells which were left to grow for the full 6 days of the assay.

The experiments were run in duplicate and the mean optical density calculated.

### Western Blotting

Cells were incubated with AS1411 for 4 days, after which cell lysates were analysed by non-reducing SDS-PAGE analysis using 4-12% NuPAGE Bis-Tris Gels (Invitrogen).

10 μg of total protein (whole cell lysate) was loaded per well as assessed by the Lowry assay (Lowry reagent available from Sigma-Aldrich, catalogue number L3540) and detected using ECL Advance western blotting kit (GE Healthcare).

Anti-nucleolin and bax antibodies were obtained from Santa Cruz and the β-actin antibody from QED.

### Results—Cytotoxicity

Cytotoxicity and cytostatic test results are shown in FIGS. 9, 10 and 11 and Table 12 below.

### Table 12: Sensitivity of Paediatric Cancer Cell Lines to AS1411.

Average IC₅₀ values are shown from at least two experiments for each cell line.

TABLE 12

| Cell line | Tumour type | IC₅₀ (nM) |
|---|---|---|
| MV4-11 | Acute myelogenous leukaemia (AML) | 2.1 |
| SUP-B15 | Acute lymphoblastic leukaemia (ALL) | 2.3 |
| R-1059-D | Osteosarcoma | 4.7 |
| A204 | Rhabdomyosarcoma | 2.6 |
| SK-N-AS | Neuroblastoma | 3.2 |

Average IC₅₀ values are shown from at least two experiments for each cell line.

From these results it can be seen that AS1411 (SEQ ID No. 1) shows activity against (i.e. reduces the cell numbers of) many paediatric cancer cell lines

### Western Blotting

Nucleolin appears as several bands: these forms are expected from the literature; no effect is observed on total cell lysate nucleolin levels. Bax is observed as both a monomer or dimer. Up-regulation of Bax is observed upon exposure to AS1411 in both cell lines; levels of β-actin were used to normalise protein concentrations. Bax is a pro-apoptotic protein involved in pore formation in mitochondrial membranes, leading to apoptosis.

### EXAMPLE 5

### Administration of AS1411 Therapy in Cancer Treatment Using an Intravenous Infusion

AS1411 is given to patients via intravenous infusion over a period of 7 days. The daily amount to be administered to the patient is calculated based on dose in mg/kg and the patient weight.

Fresh solutions are prepared on each infusion day, by diluting AS1411 drug product into 5% dextrose within an infusion bag (alternatives to dextrose include any known infusion system such as saline). Appropriate infusion bags are known to those skilled in the art. A fresh infusion bag is preferably prepared at the start of each 24-hour period. After calculation of the required dose of AS1411, an equivalent volume of dextrose should be removed from the bag, and the required dose of AS1411 added directly to the bag for a total final volume of 500 mL.

Once prepared, infusion bags containing AS1411 can be stored at +2° C. to +5° C. until administration. Drug can be prepared up to 6 hours prior to dosing.

Reconstituted AS1411 in 5% dextrose is administered at room temperature as soon as possible following reconstitution. The appropriate dose of AS1411 is administered as a 500 ml intravenous infusion. Infusion of AS1411 is as close to 24 hours as possible, accounting for changing of infusion bags, or clotting of infusion lines.

### EXAMPLE 6

### Administration of GRO in Cancer Treatment Using an Ambulatory Device

Administration of AS1411 is performed using an ambulatory device, which allows improved patient mobility. Such an administration route is useful for, for example, treatment of a patient with renal cancer.

Ambulatory devices are well-known in the art of pharmacy and medicine and a skilled person would be able to select an appropriate device. A preferred device is the Baxter FOLFusor LV10 (Baxter Parkway, Deerfield, Ill. 60015-4625, USA; FIG. 9) which been used extensively in chemotherapy treatment, is non-allergenic, and supplies product at a rate of 10 ml/hour from a 240 ml reservoir. The FOLFusor is supplied in a “burn bag” to improve patient freedom and is replaced with a fresh, filled FOLFusor each day during the treatment cycle.

In the FOLFusor, product is introduced into a central elastomeric balloon via a syringe connected to a Fill Port located on the top of the device. The balloon is filled with 240 ml of AS1411. Having filled the device, the internal pressure within the balloon then drives the flow of product from the balloon through the delivery tubing via a luer-lock connector to the catheter. The flow rate is controlled by a restriction caused by a flow restrictor in the delivery tubing.

The flow rate accuracy is +/−10% and has been calibrated by Baxter using 5% dextrose. The FOLFusor must be filled to the nominal volume (240 ml) or the flow rate is reduced. A 5 micron in-line filter removes any particulates. There is no risk of air ingress as the FOLFusor is a closed system. If the FOLFusor dispenses all product and empties, there is some risk of blood tracking back up the tubing and causing a blockage. This can be removed with a heparin flush.

Details of the administration materials are:
AS1411 Drug Product concentrate, 20 mg/ml in 20 ml vials
Baxter FOLFusor LV10 (Baxter, catalogue no. 2C4063K)
Sterile syringe with Luer Lock Fitting, 100 ml capacity (e.g. Becton-Dickinson Plastipak)
Sterile Hypodermic needle
5% dextrose solution (Viaflex Container, Baxter, e.g. catalogue no. 2B0089)
Sterile Mixing vessel (preferably around 500 ml)

### (i) AS1411 Dose Calculation

AS1411 is delivered to the clinic as a concentrate in 20 ml vials at 20 mg/ml. AS1411 is first diluted into 5% dextrose at the clinic to give a final volume of 240 ml, the ratio of 5% dextrose to AS1411 is dependent on patient weight (see Table 12, below).

### (ii) AS1411 Solution Preparation

Using Table 12 as a guide, remove the required number of AS1411 vials from the refrigerator and allow to stand at room temperature for 1 hour. Using a sterile 100 ml syringe fitted with a hypodermic needle, withdraw the required volume of AS1411 concentrate from vials and add to the sterile mixing vessel. Using the same syringe, now withdraw the required volume of 5% dextrose from the Viaflex containers and add to the AS1411 concentrate in the mixing vessel. Swirl the container contents gently to mix. Note that Steps (ii) and (iii) must be carried out in a safety cabinet.

TABLE 12
Preparation Guidelines for AS1411 at varying
patient weight for 40 mg/kg dose

| | Total g AS1411 | Volume | Volume 5% |
|---|---|---|---|
| Patient | per 24 hours at | AS1411 | dextrose |
| weight (kg) | 40 mg/kg | (ml) | (ml) |
| 60 | 2.4 | 120 | 120 |
| 65 | 2.6 | 130 | 110 |
| 70 | 2.8 | 140 | 100 |
| 75 | 3.0 | 150 | 90 |
| 80 | 3.2 | 160 | 80 |
| 85 | 3.4 | 170 | 70 |
| 90 | 3.6 | 180 | 60 |
| 95 | 3.8 | 190 | 50 |
| 100 | 4.0 | 200 | 40 |
| 105 | 4.2 | 210 | 30 |
| 110 | 4.4 | 220 | 20 |
| 115 | 4.6 | 230 | 10 |
| 120 | 4.8 | 240 | 0 |

(iii) Addition of Drug to the FOLFusor.

The AS1411/dextrose solution is added to the FOLFusor using the 100 ml syringe screwed onto the Fill Port at the top of the device. Remove the hypodermic needle from the syringe and unscrew the cap from the Fill Port on the FOLFusor and retain in the cabinet. Remove the blue cap from the end of the delivery tube attached to the FOLFusor and retain in the cabinet (removal of the blue cap will allow air to be expelled from the device during priming). Fill the syringe with 100 ml of the AS1411 dextrose solution from the container and screw the syringe onto the Fill Port; slowly push the syringe plunder to transfer the solution into the device (the central balloon will inflate). Continue this process with additional syringe filling until 240 ml of the AS1411 dextrose solution is transferred to the FOLFusor (the balloon will now be fully inflated). Allow the drug solution to drip from the end of the delivery tube before replacing the blue cap.

### (iv) Connecting to the Catheter and Patient.

Now remove the filled FOLFusor from the safety cabinet. Using aseptic technique, remove the blue, cap from the end of the delivery tube and attach to the catheter via the luer lock fitting. Allow drug solution to drip from the catheter before attaching to the patient.

### (v) Guidelines on Use.

The FOLFusor is then placed in a pouch attached to the patient's waist (such as in a “burn bag” which refer to a pouch attached to a belt that can be worn around the waist). The FOLFusor should be kept at roughly the same height as the entry port into the patient. The flow rate decreases by 0.5% per 2.5 cm below this level, and increases by 0.5% per 2.5 cm above this level. Temperature and viscosity also impact the flow rate. A reduced temperature increases the viscosity and decreases the flow rate. A higher temp reduces the viscosity and increases the flow rate. 33.3° C. is the assumed temperature in the burn bag.

### EXAMPLE 7

### Preferred Pharmaceutical Formulations and Modes and Doses of Administration

The polynucleotides and chemotherapeutics of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The polynucleotides and chemotherapeutics of the present invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for administration. A device which delivers a pulsed electric field to cells increases the permeability of the cell membranes to the drug, resulting in a significant enhancement of intracellular drug delivery.

Polynucleotides and chemotherapeutics of the invention can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of administration is the ReGel injectable system that is thermosensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Polynucleotides and chemotherapeutics of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient (Derossi et al., 1998, Trends Cell Biol., 8, 84-87).

Preferably, the pharmaceutical formulation of the present invention is a unit dosage containing a daily dose or unit, daily sub-dose or an appropriate fraction thereof, of the active ingredient.

The polypeptides, polynucleotides and antibodies of the invention can be administered by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the polypeptides, polynucleotides and antibodies of the invention can be administered alone but will generally be administered in admixture with a suitable pharmaceutical excipient diluent or carrier selected with regard to the intended route of administration and standard pharmaceutical practice.

The polypeptides, polynucleotides and antibodies of the invention can also be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Generally, in humans, continuous intravenous administration of the polynucleotides and chemotherapeutics of the invention is the preferred route.

For veterinary use, the polynucleotides and chemotherapeutics of the invention are administered as a suitably acceptable formulation in accordance with normal veterinary practice and the veterinary surgeon will determine the dosing regimen and route of administration which will be most appropriate for a particular animal.

The formulations of the pharmaceutical compositions of the invention may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question.

### EXAMPLE 8

### Exemplary Pharmaceutical Formulations

Whilst it is possible for polynucleotides and chemotherapeutics of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen-free.

The following examples illustrate pharmaceutical formulations according to the invention in which the active ingredient is a polynucleotide and/or chemotherapeutic of the invention.

### EXAMPLE 8A

### Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |

| | |
|---|---|
| pH adjusted to | 7.5 |

### EXAMPLE 8B

### Capsule Formulations

### Formulation A

A capsule formulation is prepared by admixing the ingredients of Formulation D in Example C above and filling into a two-part hard gelatin capsule. Formulation B (infra) is prepared in a similar manner.

Formulation B

| | mg/capsule |
|---|---|
| Active ingredient | 250 |
| Lactose B.P. | 143 |
| Sodium Starch Glycolate | 25 |
| Magnesium Stearate | 2 |
| | 420 |

Formulation C

| | mg/capsule |
|---|---|
| Active ingredient | 250 |
| Macrogol 4000 BP | 350 |
| | 600 |

Capsules are prepared by melting the Macrogol 4000 BP, dispersing the active ingredient in the melt and filling the melt into a two-part hard gelatin capsule.

Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

### Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| Active ingredient | 250 |
| Microcrystalline Cellulose | 125 |
| Lactose BP | 125 |
| Ethyl Cellulose | 13 |
| | 513 |

### EXAMPLE 8C

### Injectable Formulation

| | | |
|---|---|---|
| Active ingredient | 0.200 | g |
| Sterile, pyrogen free phosphate buffer (pH 7.0) to | 10 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

Alternatively, the formulation may contain the following:
Potassium phosphate dibasic USP Quality (EMD Chemicals Inc, New Jersey 08027, USA) to pH 7.4;
Potassium phosphate monobasic USP Quality (EMD Chemicals Inc) to pH 7.4;
Water for Injection to 20 ml;
AS1411 400 mg

The weights of these materials used in each batch will depend on batch size. For example, the following could be used to give a batch size yielding approximately 1370 vials containing 20 ml at 20 mg/ml AS1411:
AS1411 528.5 g;
Potassium phosphate dibasic 39.8 g;
Potassium phosphate monobasic 8.2 g;
Water for Injection to 28339.8 g;
the formulation is mixed with 5% dextrose (Baxter) at the clinic.

### EXAMPLE 8D

### Intramuscular Injection

| | | |
|---|---|---|
| Active ingredient | 0.20 | g |
| Benzyl Alcohol | 0.10 | g |
| Glucofurol 75 ® | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### EXAMPLE 8E

### Syrup Suspension

| | | |
|---|---|---|
| Active ingredient | 0.2500 | g |
| Sorbitol Solution | 1.5000 | g |
| Glycerol | 2.0000 | g |
| Dispersible Cellulose | 0.0750 | g |
| Sodium Benzoate | 0.0050 | g |
| Flavour, Peach 17.42.3169 | 0.0125 | ml |
| Purified Water q.s. to | 5.0000 | ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### EXAMPLE 8F

### Suppository

| | mg/suppository |
|---|---|
| Active ingredient (63 μm)* | 250 |
| Hard Fat, BP (Witepsol H15 - Dynamit Nobel) | 1770 |
| | 2020 |

*The active ingredient is used as a powder wherein at least 90% of the particles are of 63 μm diameter or less.

One fifth of the Witepsol H15 is melted in a steam jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### EXAMPLE 8G

### Pessaries

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### EXAMPLE 8H

### Creams and Ointments

Described in Remington, The Science and Practise of Pharmacy, 19^{th} ed., The Philadelphia College of Pharmacy and Science, ISBN 0-912734-04-3.

### EXAMPLE 8I

### Microsphere Formulations

The compounds of the invention may also be delivered using microsphere formulations, such as those described in Cleland (1997, Pharm. Biotechnol. 10:1-43; and 2001, J. Control. Release 72:13-24).

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A pharmaceutical composition comprising a G rich oligonucleotide having the sequence of one of SEQ ID NOs: 1 to 18 and an anthracycline in conjunction with a pharmaceutically acceptable excipient, diluent or carrier.

2. A pharmaceutical composition of claim 1, wherein the anthracycline is doxorubicin.

3. A pharmaceutical composition as claimed in claim 1 wherein the G-rich oligonucleotide has the sequence of SEQ ID NO: 1.

4. A pharmaceutical composition as claimed in claim 1 wherein the G-rich oligonucleotide has a 3′ end and a 5′ end, and one or both of the 3′ and 5′ ends have been modified to alter a property of the G-rich oligonucleotide.

5-10. (canceled)

11. A method for inhibiting the proliferation of malignant, dysplastic, and/or hyperproliferative cells in a subject, said method comprising administering to the subject a therapeutically effective amount of a G-rich oligonucleotide having the sequence of one of SEQ ID NOs: 1 to 18 in combination with an anthracycline.

12. A method of claim 11, wherein the anthracycline is doxorubicin.

13. A method as claimed in claim 11 wherein the administration of the G-rich oligonucleotide precedes administration of the anthracycline.

14. A method as claimed in claim 11 wherein the administration of the anthracycline precedes administration of the G-rich oligonucleotide.

15. A method as claimed in claim 11 wherein both the G-rich oligonucleotide and the anthracycline are administered simultaneously.

16. A method as claimed in claim 11 wherein the G-rich oligonucleotide has the sequence of SEQ ID NO: 1.

17. A method as claimed in claim 16 wherein the G-rich oligonucleotide has a 3′ end and a 5′ end, and one or both of the 3′ and 5′ ends have been modified to alter a property of the G-rich oligonucleotide.

18. A method as claimed in claim 11, wherein the malignant, dysplastic, and/or hyperproliferative cells are associated with at least one of the following disorders: acute myelogenous leukaemia, acute myeloid leukaemia (AML), acute lymphoblastic leukaemia (ALL), chronic myelogenous leukemia (CML), Wilm's tumour, neuroblastoma, soft tissue and bone sarcomas, breast carcinoma, ovarian carcinoma, bladder carcinoma, pancreas carcinoma, thyroid carcinoma, gastric cancer, renal cancer, malignant lymphoma, bronchiogenic carcinoma, basal cell carcinoma, melanoma, acute promyelocytic leukaemia, myelodysplastic syndrome, chronic lymphocytic leukemia, rhabdomyosarcoma; osteosarcoma; medulloblastoma; craniopharyngioma; retinoblastoma; Ewing's sarcoma; lymphomas; non-Hodgkin's lymphoma; and Hodgkin's lymphoma and solid tumours.

19. A method for treating a disease characterised by malignant, dysplastic, and/or hyperproliferative cells comprising exposing the malignant, dysplastic, and/or hyperproliferative cells to a combination of a G-rich oligonucleotide having SEQ ID NO: 1 and doxorubicin; wherein the G-rich oligonucleotide and doxorubicin are administered in combination with one another and the malignant, dysplastic, and/or hyperproliferative cells are associated with at least one of the following disorders: acute myelogenous leukaemia, acute myeloid leukaemia (AML), acute lymphoblastic leukaemia (ALL), chronic myelogenous leukemia (CML), Wilm's tumour, neuroblastoma, soft tissue and bone sarcomas, breast carcinoma, ovarian carcinoma, bladder carcinoma, pancreas carcinoma, thyroid carcinoma, gastric cancer, renal cancer, malignant lymphoma, bronchiogenic carcinoma, basal cell carcinoma, melanoma, acute promyelocytic leukaemia, myelodysplastic syndrome, chronic lymphocytic leukemia, rhabdomyosarcoma; osteosarcoma; medulloblastoma; craniopharyngioma; retinoblastoma; Ewing's sarcoma; lymphomas; non-Hodgkin's lymphoma; and Hodgkin's lymphoma and solid tumours.

20. A method as claimed in claim 19 wherein the administration of the G-rich oligonucleotide precedes administration of doxorubicin.

21. A method as claimed in claim 19 wherein administration of the doxorubicin precedes administration of the G-rich oligonucleotide.

22. A method as claimed in claim 19 wherein both the G-rich oligonucleotide and doxorubicin are administered simultaneously.

23. (canceled)

24. A method as claimed in claim 19 wherein the G-rich oligonucleotide has a 3′ end and a 5′ end, and one or both of the 3′ and 5′ ends have been modified to alter a property of the G-rich oligonucleotide.

25. (canceled)

26. A method as claimed in claim 19 wherein the disorder is acute myelogenous leukaemia, acute myeloid leukaemia (AML), or a lymphoma.

27-46. (canceled)

47. A method of claim 18, wherein the disorder is selected from: acute myelogenous leukaemia; acute myeloid leukaemia (AML); acute lymphoblastic leukaemia (ALL), chronic myelogenous leukemia (CML), lymphomas, non-Hodgkin's lymphoma, Burkitt's lymphoma, acute promyelocytic leukaemia, myelodysplastic syndrome, chronic lymphocytic leukemia, and Hodgkin's lymphoma.

48. The pharmaceutical composition of claim 1, wherein the anthracycline is selected from the group consisting of doxorubicin, daunorubicin, epirubicin, idarubicin, and nemorubicin.

49. A method for treating cancer in a subject comprising administering to the subject a therapeutically effective amount of a G-rich oligonucleotide having the sequence of one of SEQ ID NOs: 1 to 18 in combination with an anthracycline selected from doxorubicin, daunorubicin, epirubicin, idarubicin, and nemorubicin.

50. The method of claim 49 wherein the cancer is selected from the group of acute myelogenous leukaemia, acute myeloid leukaemia (AML), acute lymphoblastic leukaemia (ALL), chronic myelogenous leukemia (CML), lymphomas, non-Hodgkin's lymphoma, acute promyelocytic leukaemia, myelodysplastic syndrome, chronic lymphocytic leukemia, and Hodgkin's lymphoma.

51. The method of claim 11, wherein the anthracycline is selected from daunorubicin, epirubicin, idarubicin, and nemorubicin.
